(19) United States
(12) Patent Application Publication
(10) Pub. No.: US 2025/0213752 A1
(43) Pub. Date: Jul. 3, 2025

(54) DUAL POROSITY MEMBRANE

(71) Applicant: Northwestern University, Evanston, IL (US)

(72) Inventors: Guillermo A. AMEER, Evanston, IL (US); Jonathan RIVNAY, Evanston, IL (US); Huifeng WANG, Evanston, IL (US)

(21) Appl. No.: 18/866,846

(22) PCT Filed: May 18, 2023
(86) PCT No.: PCT/US2023/022761

§ 371 Date: Nov. 18, 2024

Related U.S. Application Data
(60) Provisional application No. 63/343,420, filed on May 18, 2022.

(51) Int. Cl. A61L 27/56; A61L 27/18; A61L 27/54; B32B 27/40; C12N 5/00

(52) U.S. Cl. CPC A61L 27/56; A61L 27/18; A61L 27/54; B32B 27/40; C12N 5/0068; B32B 2250/02; B32B 2264/2031

(57) ABSTRACT

Provided herein are compositions comprising dual-porosity membranes an microporous outer layer and a nanoporous inner layer, and methods of use thereof. In particular, polycarbonate-based polyurethane bilayer membranes are formed in the presence of pore-forming agents that allow for differ pore sizes to be achieved in inner and outer layers. The dual-porosity membranes herein find use in encapsulating therapeutic cells for transplantation.

### CROSS-REFERENCE TO RELATED APPLICATIONS

This application claims the benefit of U.S. Provisional Patent Application No. 63/343,420, filed on May 18, 2022, which is incorporated by reference herein.

### STATEMENT REGARDING FEDERAL FUNDING

This invention was made in part with government under FA8650-21-2-7119 awarded by the Department of the Air Force, Air Force Research Laboratory. The government has certain rights in the invention.

### FIELD

Provided herein are compositions comprising dual-porosity membranes an microporous outer layer and a nanoporous inner layer, and methods of use thereof. In particular, polycarbonate-based polyurethane dual-porosity membranes are formed in the presence of pore-forming agents that allow for differ pore sizes to be achieved in inner and outer layers. The dual-porosity membranes herein find use in encapsulating therapeutic cells for transplantation.

### BACKGROUND

Transplanted therapeutic cells are subject to the host's immune response and must be protected. Therefore, therapeutic cells require a membrane with selective transport functions that are immunoprotective yet conducive to maintaining cell functions and therapeutic delivery.

### SUMMARY

Provided herein are compositions comprising dual-porosity membranes an microporous outer layer and a nanoporous inner layer, and methods of use thereof. In particular, polycarbonate-based polyurethane dual-porosity membranes are formed in the presence of pore-forming agents that allow for differ pore sizes to be achieved in inner and outer layers. The dual-porosity membranes herein find use in encapsulating therapeutic cells for transplantation.

In some embodiments, provided herein are compositions comprising a dual-porosity membrane comprising an microporous outer layer and a nanoporous inner layer. In some embodiments, the microporous outer layer has pore sizes of 1-50 μm in size (e.g., 1 μm, 2 μm, 5 μm, 10 μm, 20 μm, 50 μm, or ranges therebetween). In some embodiments, the nanoporous inner layer has pore sizes of 100-1000 nm in size (e.g., 100 nm, 200 nm, 500 nm, 1000 nm, or ranges therebetween). In some embodiments, a base material comprises the microporous outer layer and the nanoporous inner layer. In some embodiments, the base material is a polyurethane. In some embodiments, the base material is a polycarbonate-based polyurethane.

In some embodiments, provided herein are methods of fabricating a dual-porosity membrane comprising: (a) combining a first solution of an elastomeric base material dissolved in an organic solvent with a first pore-forming agent to form a first pore-forming mixture; (b) allowing the organic solvent to evaporate from the first pore-forming mixture thereby forming a first layer of membrane comprising the elastomeric base material with the first pore-forming agent embedded therein; (c) combining a second solution of the elastomeric base material dissolved in an organic solvent with a second pore-forming agent to form a second pore-forming mixture; (d) layering the second pore-forming mixture onto the first membrane; (e) allowing the organic solvent to evaporate from the second pore-forming mixture thereby forming a second layer of membrane comprising the elastomeric base material with the second pore-forming agent embedded therein; and (f) washing with an wash solution to wash the first and second pore-forming agents from the first and second layers of membrane, thereby producing a dual-porosity membrane. In some embodiments, the elastomeric base material is a polyurethane. In some embodiments, the polyurethane is a polycarbonate-based polyurethane. In some embodiments, the organic solvent has a relative polarity of less then 0.5. In some embodiments, the organic solvent is tetrahydrofuran (THF). In some embodiments, the first and second pore-forming agents are insoluble (or extremely low solubility) in the organic solvent. In some embodiments, the first and second pore-forming agents are soluble (or extremely high solubility) in the wash solution. In some embodiments, the first pore-forming agent is a salt. In some embodiments, the first pore-forming agent is ammonium persulfate. In some embodiments, the second pore-forming agent is a polyethylene glycol. In some embodiments, the second pore-forming agent is a PEG 200. In some embodiments, the second pore-forming agent is an end-capped PEG. In some embodiments, the wash solution is an aqueous solution. In some embodiments, the first and second pore-forming agents are washed from the membrane in a single step. In some embodiments, the first pore-forming agents are washed from the membrane before fabrication of the second layer. In some embodiments, provided herein are dual-porosity membranes fabricated by the methods herein.

In some embodiments, provided herein are methods comprising culturing cells on a dual-porosity membrane described herein.

In some embodiments, provided herein are methods comprising administering to a subject cells on or within a dual-porosity membrane described herein.

### BRIEF DESCRIPTION OF THE DRAWINGS

FIG. 1. Fabrication of dual-porosity membrane The outer microporous layer and nanoporous layer were formed by mixing 20% ammonium persulfate and PEG 200 with 5% PU solution in THF, respectively. After THE completely evaporated, the dual-porosity membrane was washed in isopropanol and water solution (1:1) overnight.

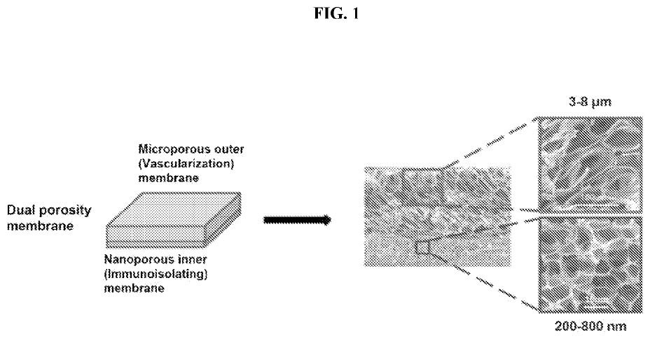

FIG. 2. T cell and glucose permeability test. Jurkat cells and glucose were added into transwell insert. After 2 h and 24 h, the cells and glucose permeate through the membranes were measured by alamar Blue and colorimetric assay, respectively.

FIG. 3. SEM images of dual-porosity membrane.

FIG. 4A-C. Cytotoxicity tests of dual-porosity membrane using (a) and (b) extraction method and (c) direct contact method.

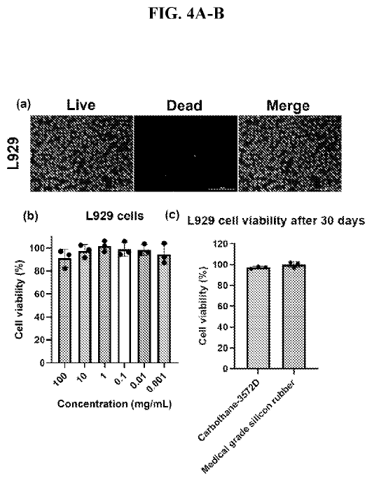

FIG. 5A-B. (a) T cell and (b) glucose permeability test.

FIG. 6A-D. (a) Weight, (b) diameter and (c) thickness change after 60-day implantation. (d) SEM images of dual-porosity membrane after 60-day implantation.

FIG. 7A-B. (a) Digital images of the fibrous capsule tissue. (b) Quantification of fibrous capsule thickness.

FIG. 8A-C. (a) Representative immunofluorescence images of CD31 and α-smooth muscle actin (α-SMA). (b) Relative fluorescence intensity from the immunofluorescence images for CD31 and α-SMA.

### DEFINITIONS

As sued herein, the term “polymer” a material comprising a plurality of linked monomer units. The term “polymer” as used herein includes, but is not limited to, homopolymers (all the same monomer units), copolymers (two different monomer units), terpolymers (three different monomer units), etc. The term “polymer” as used herein also includes impact, block, graft, random, and alternating copolymers.

As used herein, the term “thermoplastic” refers to a plastic polymer material that softens and hardens reversibly on heating and cooling. Thermoplastic polymers encompass thermoplastic elastomers.

As used herein, the term “elastomer” refers to a polymer exhibiting viscoelasticity (e.g., “rubber-like elasticity”).

### DETAILED DESCRIPTION

Provided herein are compositions comprising dual-porosity membranes an microporous outer layer and a nanoporous inner layer, and methods of use thereof. In particular, polycarbonate-based polyurethane dual-porosity membranes are formed in the presence of pore-forming agents that allow for differ pore sizes to be achieved in inner and outer layers. The dual-porosity membranes herein find use in encapsulating therapeutic cells for transplantation.

In order to better encapsulate therapeutic cells for transplantation, protect them from host's immune response and maintain their long-term survival, the dual-porosity membranes herein were fabricated with an outer microporous layer for vascularization and an inner nanporous layer for immunoisolation. Briefly, the invention provides fabrication of dual-porosity membrane using agent leaching strategy, which dissolve polyurethane (PU) base material and water-soluble pore forming agent in organic solvent and form a film in the mold. After washed out pore forming agent in water, the porous membrane was fabricated.

Previous studies have shown that the polymeric porous membrane with pore sizes <1 μm can prevent the infiltration of immune cells. Furthermore, blood vessels that can provide nutrients and oxygen to cells should be in close proximity to the membrane. There is evidence that macroporous membranes can support vascularization. Experiments were conducted during development of embodiments herein to develop a membrane that would prevent immune attack yet promote vascularization to support the needs of cell factories and assure rapid delivery and systemic uptake of therapeutic molecules. In some embodiments, provided herein are dual-porosity membranes fabricated from medical grade polyurethane (PU) as a base material and PEG and ammonium sulfate as pore forming agents.

In some embodiments, the membranes herein find use in islets transplantation for treatment of Type I diabetes. In some embodiments, the membranes herein find use with engineered cells that can release therapeutic agents, such as therapeutic peptides, cytokines and growth factors for treatment of diseases such as cancer, cardiovascular diseases and obesity.

Most existing technologies to create dual layer membrane for cell transplantation are laminate two membranes with different pore size, which often have delamination problems resulting in leakage cells. The dual-porosity membranes herein are a single membrane with two pore size on each side, which doesn't require lamination. Another existing technology to fabricate dual-porosity membrane for cell transplantation is using electrospinning techniques, which usually include toxic chemicals and high voltage electricity. The methods herein to fabricate dual-porosity membranes utilize an agent leaching method, which didn't involve any toxic chemicals or high-power usage and ease of fabrication. Moreover, the porous membranes generated by electrospinning techniques exhibit variation from batch to batch. The methods herein eliminate variation and produce a consistent dual membrane product.

In a particular embodiment, a dual-porosity membrane was fabricated by using medical grade polycarbonate-based polyurethane (PU) as a base material and end-capped polyethylene glycol (PEG) and ammonium sulfate salt as pore forming agents. SEM images demonstrate that the exemplary dual-porosity membrane was successfully created with a 750 nm of nanoporous layer and a 11.7 μm of microporous layer. Cell viability of the membrane was studied by both extraction method and direct contact method according to ISO 10093 standards for biocompatibility test of medical devices. The results indicate that the dual-porosity membrane is safe to ARPE-19 cells for 30 days. The exemplary dual-porosity membrane can effectively inhibit T cell penetrating through it, whereas it didn't affect glucose permeability, which 79% glucose permeate through those membranes after 24 hours. The membrane is stable and nonbiodegradable for 60 days in vivo in a mouse model, which there is no obvious difference in weight, size and pore structure before and after subcutaneous implantation. The membrane can mitigate the foreign body response, which demonstrated >60% reduction in fibrous capsule thickness relative to PDMS, a standard material currently used in implants, and an approximate 300% increase in vascularization at the membrane interface relative to PDMS.

In some embodiments, the dual-porosity membranes herein comprise one or more base materials with pores formed therein. In some embodiments, the base material is a polymer, elastomer, and/or thermoplastic. In some embodiments, the base material is a polyurethane (e.g., polycarbonate-based polyurethane, polyether-based polyurethane, etc.) or thermoplastic polymer variant of polyurethane.

A polyurethane is typically produced by reacting an isocyanate with a polyol. Since a polyurethane contains two types of monomers, which polymerize one after the other, they are classed as alternating copolymers. Both the isocyanates and polyols used to make a polyurethane contain two or more functional groups per molecule. Polyurethanes are produced by reacting diisocyanates with polyols, often in the presence of a catalyst or upon exposure to ultraviolet light. Common catalysts include tertiary amines, such as DABCO, or metallic soaps, such as dibutyltin dilaurate.

Isocyanates used to make polyurethane have two or more isocyanate groups on each molecule. The most commonly used isocyanates are the aromatic diisocyanates, toluene diisocyanate (TDI) and methylene diphenyl diisocyanate, (MDI). Aliphatic and cycloaliphatic isocyanates may also be used.

Polyols are polymers and have on average two or more hydroxyl groups per molecule. They can be converted to polyether polyols co-polymerizing ethylene oxide and propylene oxide with a suitable polyol precursor. Polyester polyols are made by the polycondensation of multifunctional carboxylic acids and polyhydroxyl compounds. Polyols including polycarbonate polyols, polyether polyols, polycaprolactone polyols, polybutadiene polyols, and polysulfide polyols can be used in the production of polyurethanes.

In addition to polyols and diisocyanates, polyurethanes may also include one or more chain extenders and/or cross linkers, low molecular weight hydroxyl and amine terminated compounds that affect the structure and properties of the polyurethane. Examples of chain extenders and cross linkers that may find use in the polyurethanes herein include ethylene glycol, diethylene glycol, triethylene glycol, tetraethylene glycol, propylene glycol, dipropylene glycol, tripropylene glycol, 1,3-propanediol, 1,3-butanediol, 1,4-butanediol, neopentyl glycol, 1,6-hexanediol, 1,4-cyclohexanedimethanol, HQEE, ethanolamine, diethanolamine, methyldiethanolamine, phenyldiethanolamine, glycerol, trimethylolpropane, 1,2,6-hexanetriol, Triethanolamine, pentaerythritol, N,N,N′,N′-tetrakis, (2-hydroxypropyl), Ethylenediamine, diethyltoluenediamine, dimethylthiotoluenediamine, etc.

In some embodiments, the base material is a polycarbonate-based polyurethane, such as those described in Špírková et al. (European Polymer Journal, Volume 47, Issue 5, 2011, Pages 959-972; incorporated by reference in its entirety). In some embodiments, a polycarbonate-based polyurethane is prepared from a polycarbonate diol and a diisocyanate (e.g., hexamethylene diisocyanate), optionally with one or more chain extenders (e.g., butane-1,4-diol). Examples of polycarbonate-based polyurethanes are described in Wu et al. Appl. Sci. 2021, 11(12), 5359 and Selvakumar et al. J Biomed Nanotechnol. 2015 Feb;11(2):291-305; incorporated by reference in their entireties.

In some embodiments, pores are introduced into the base material of the dual-porosity membranes herein through he inclusion of pore-forming agents. In some embodiments, the pore-forming agents herein are water soluble but insoluble (or extremely low solubility) in the organic solvent used in the membrane fabrication.

Examples of suitable pore-forming agents include salts such as sodium chloride, aluminum chloride, sodium phosphate, sodium bicarbonate, ammonium persulfate, magnesium sulfate, potassium iodide, copper sulfate, calcium chloride, potassium permanganate, sodium acetate, potassium nitrate, calcium carbonate, calcium oxide, etc.

Other pore-forming agents include polyethylene glycols (PEGs), such as PEGS with a molecular weights ranging from 300 g/mol to 10,000,000 g/mol (e.g., 300 g/mol, 500 g/mol, 750 g/mol, 1,000 g/mol, 1,500 g/mol, 2,000 g/mol, 3,000 g/mol, 5,000 g/mol, 10,000 g/mol, 20,000 g/mol, 50,000 g/mol, 100,000 g/mol, 200,000 g/mol, 500,000, g/mol, 1,000,000 g/mol, 2,000,000 g/mol, 5,000,000 g/mol, 10,000,000 g/mol, or ranges therebetween.). PEG pore-forming agents may be branched PEGs or start PEGs. In some embodiments, PEG pore-forming agents may be modified, such as by end capping (e.g., PEG end-capped with ethylene oxide (EO) and propylene oxide (PO). Any suitable PEG may find use as a pore-forming agent in embodiments herein.

In some embodiments, various solvents may find use in the fabrication of the dual-porosity membranes herein. In some embodiments, a solvent is selected that comprises one or more (e.g., all) of the following characteristics: base material is soluble in the solvent (under the fabrication conditions), port-forming material(s) are insoluble in the solvent (under the fabrication conditions), easily evaporated (under the fabrication conditions), etc. Suitable solvents include for certain embodiments herein may be selected from hexane, p-xylene, toluene, benzene, ether, methyl t-butyl ether (MTBE), diethylamine, dioxane, chlorobenzene, tetrahydrofuran (THF), ODCB (orthodichlorobenzene), ethyl acetate, dimethoxyethane (DME; glyme), pyridine, methylene chloride, HMPT, 1,2-dichloroethane, DMPU, acetone, dimethylformamide (DMF), t-butyl alcohol, sulfolane, dimethylsulfoxide (DMSO), acetonitrile, nitromethane, etc.

In some embodiments, various wash solutions may be employed in the fabrication of the dual-porosity membranes herein. In some embodiments, a wash solution is selected that with dissolve the pore-forming agent without disrupting the base material membrane. In some embodiments, a wash solution is an aqueous solution. In some embodiments, a wash solution comprises water. In some embodiments, a wash solution comprises a polar or hydrophile solvent. In some embodiments, a wash solution comprises one or more of water, isopropanol, benzyl alcohol, acetic acid, ethanol, methanol, ethylene glycol, trifluoroethanol, hexafluoroisopropanol, etc.

In some embodiments, the dual-porosity membrane is fabricated by first fabricating a first layer of the membrane having a first porosity, and then fabricating a second layer of the membrane on the first layer, the second layer having a second porosity. In some embodiments, the first and second layers comprise the same base material(s). In other embodiments, the two layers comprise two different base compositions (each comprising one or more base materials). In some embodiments, the first and second layers are fabricated using the same or different solvents. the first and second layers are fabricated using different pore-forming agents (or combinations of pore forming agents), different concentrations of pore-forming agents, etc., thereby resulting in different size, shape, density, etc. of pores for the two layers.

In some embodiments, a first layer of the dual-porosity membrane is fabricated by dissolving the base material(s) (e.g., polyurethane-based material) in solvent (e.g., THF), and combining it with one or more pore-forming agents. The resulting mixture is placed on a surface (e.g., flat surface) and the solvent is allowed to evaporate. In some embodiments, elevated temperature (e.g., 40° C., 45° C., 50° C., 55° C., 60° C., 65° C., 70° C., 75° C., 80° C., 85° C., 90° C., or ranges therebetween) and/or reduced pressure (e.g., less than 1 atm) is applied to speed evaporation. Complete evaporation of the solvent results in the formation of a membrane layer of the base material(s) having the pore-forming agent embedded therein. In some embodiments, the first layer is washed one or more times (with one or more different wash solutions) to remove the embedded pore-forming agent(s). In some embodiments, the first layer is washed before formation of the second layer. In other embodiments, the pore-forming agent(s) are allowed to remain in the first layer while the second layer is fabricated thereon.

In some embodiments, a second layer of the dual-porosity membrane is fabricated by dissolving the base material(s) (e.g., polyurethane-based material) in solvent (e.g., THF), and combining it with one or more pore-forming agents. The resulting mixture is placed on a the already fabricated first layer and the solvent is allowed to evaporate. In some embodiments, elevated temperature (e.g., 40° C., 45° C., 50° C., 55° C., 60° C., 65° C., 70° C., 75° C., 80° C., 85° C., 90° C., or ranges therebetween) and/or reduced pressure (e.g., less than 1 atm) is applied to speed evaporation. Complete evaporation of the solvent results in the formation of a second membrane layer of the base material(s) atop the first layer and having the pore-forming agent embedded therein. In some embodiments, the second layer is washed one or more times (with one or more different wash solutions) to remove the embedded pore-forming agent(s).

In some embodiments, depending on the pore-forming agent(s) selected, the base material(s), solvent, and conditions used, a membrane layer may comprise pores ranging from 50 nm to 50 μm is diameter (e.g., 50 nm, 75 nm, 100 nm, 200 nm, 500 nm, 1 μm, 2 μm, 5 μm, 10 μm, 20 μm, 50, μm, or ranges therebetween). In some embodiments, a dual-porosity membrane comprises a first layer that is microporous (e.g., pores of 1 to 50 μm) and a second layer that is nanoporous (e.g., pores between 50 and 1000 nm), but membranes having other combinations of nanopores and micropores are within the scope herein.

In some embodiments, the dual-porosity membranes herein find use in cell growth and/or transplantation. For example, the membrane may be used to cover (e.g., in a chamber) or encapsulate cells for culture and/or transplantation. In some embodiments, a system is provide comprising cells and the dual-porosity membranes herein. In some embodiments, methods are provided comprising transplanting cells on or encapsulated within the dual-porosity membranes herein into a subject.

### EXPERIMENTAL

### Fabrication of Dual-Porosity Membrane

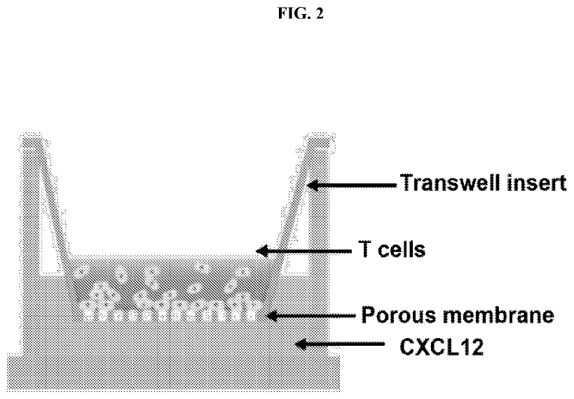

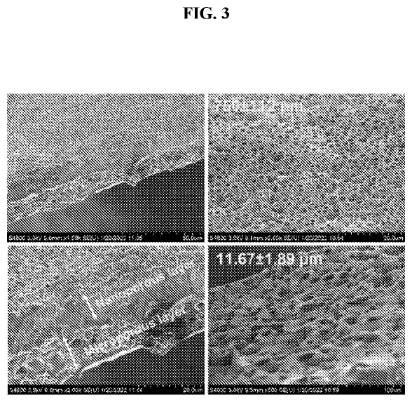

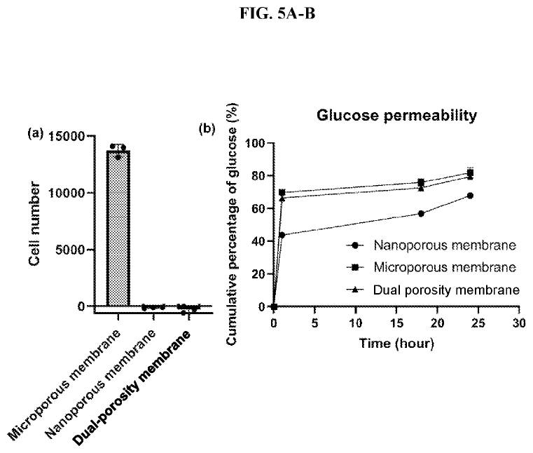

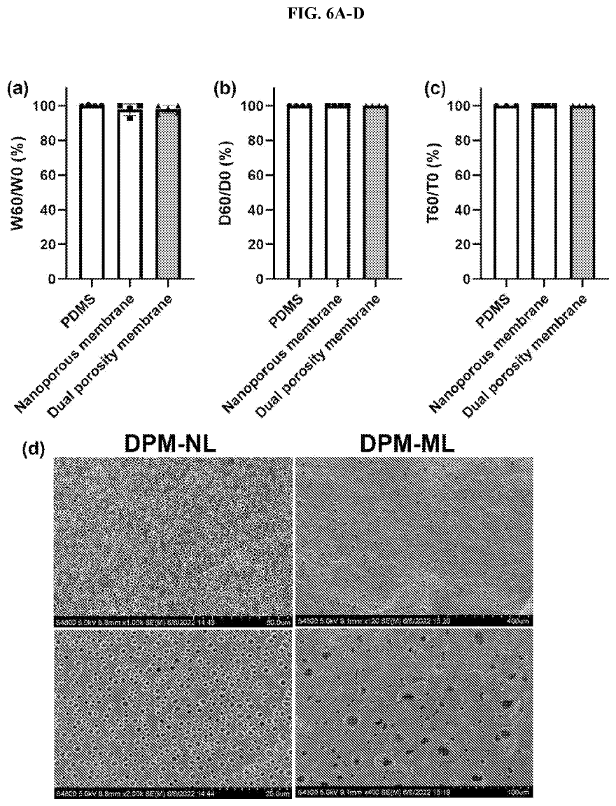

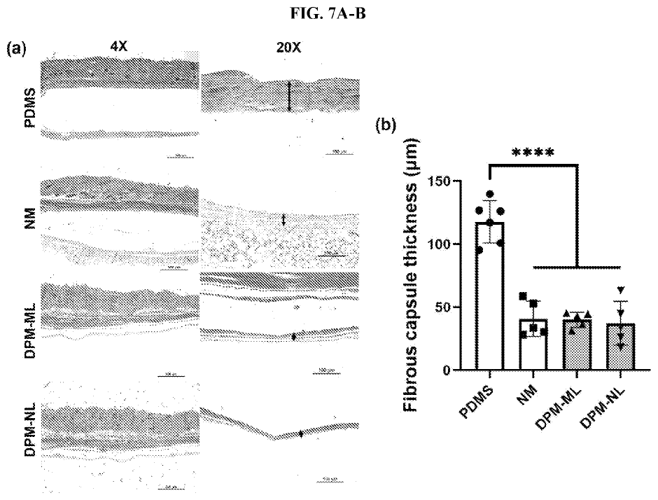

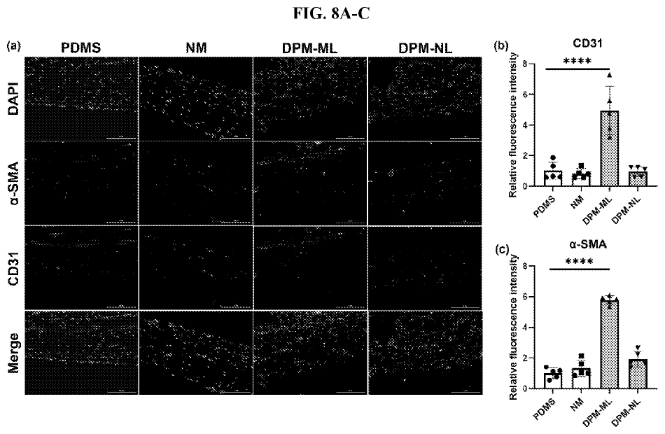

The dual-porosity membrane was fabricated by using polyurethane (PU) as a base material and end-cap polyethylene glycol and ammonium sulfate as pore forming agents. The outer microporous layer was first formed by mixing 20% ammonium persulfate with 5% PU solution in THF. After THE completely evaporated, the inner nanoporous layer was formed on top of microporous membrane by mixing 20% PEG 200 dissolved in 5% PU solution. Subsequently, the dual-porosity membrane was washed in isopropanol and water solution (1:1) for 48 hours and then washed in water for 48 hours. The resulting dual-porosity membrane demonstrated outer microporous layer with 11.7 μm pore structure and inner nanoporous layer with 750 nm pore structure. (FIG. 3) The cytotoxicity test of the dual-porosity membrane was studied by both extraction method and direct contact method according to ISO 10093 standards for biocompatibility test of medical devices. The results indicate that the dual-porosity membrane is safe to L929 cells for 30 days. (FIG. 2) The dual-porosity membrane can effectively inhibit T cell penetrating through it, whereas it didn't affect glucose permeability, which 79% glucose permeate through those membranes after 24 hours (FIG. 5). The membrane is stable and nonbiodegradable for 60 days in vivo in a mouse model, which there is no obvious difference in weight, size and pore structure before and after subcutaneous implantation. (FIG. 6) The membrane can mitigate the foreign body response, which demonstrated >60% reduction in fibrous capsule thickness relative to PDMS, a standard material currently used in implants, and an approximate 300% increase in vascularization at the membrane interface relative to PDMS. (FIGS. 7 and 8)

1. A composition comprising a dual-porosity membrane comprising an microporous outer layer and a nanoporous inner layer.

2. The composition of claim 1, wherein the microporous outer layer has pore sizes of 1-50 um in size.

3. The composition of claim 1, wherein the nanoporous inner layer has pore sizes of 100-1000 nm in size.

4. The composition of claim 1, wherein a base material comprises the microporous outer layer and the nanoporous inner layer.

5. The composition of claim 4, wherein the base material is a polyurethane.

6. The composition of claim 5, wherein the base material is a polycarbonate-based polyurethane.

7. A method of fabricating a dual-porosity membrane comprising:
(a) combining a first solution of an elastomeric base material dissolved in an organic solvent with a first pore-forming agent to form a first pore-forming mixture;
(b) allowing the organic solvent to evaporate from the first pore-forming mixture thereby forming a first layer of membrane comprising the elastomeric base material with the first pore-forming agent embedded therein;
(c) combining a second solution of the elastomeric base material dissolved in an organic solvent with a second pore-forming agent to form a second pore-forming mixture;
(d) layering the second pore-forming mixture onto the first membrane;
(e) allowing the organic solvent to evaporate from the second pore-forming mixture thereby forming a second layer of membrane comprising the elastomeric base material with the second pore-forming agent embedded therein; and
(f) washing with an wash solution to wash the first and second pore-forming agents from the first and second layers of membrane, thereby producing a dual-porosity membrane.

8. The method of claim 7, wherein the elastomeric base material is a polyurethane.

9. The method of claim 8, wherein the polyurethane is a polycarbonate-based polyurethane.

10. The method of claim 7, wherein the organic solvent has a relative polarity of less then 0.5.

11. The method of claim 7, wherein the organic solvent is tetrahydrofuran (THF).

12. The method of claim 7, wherein the first and second pore-forming agents are insoluble (or extremely low solubility) in the organic solvent.

13. The method of claim 12, wherein the first and second pore-forming agents are soluble (or extremely high solubility) in the wash solution.

14. The method of claim 7, wherein the first pore-forming agent is a salt.

15. The method of claim 14, wherein the first pore-forming agent is ammonium persulfate.

16. The method of claim 7, wherein the second pore-forming agent is a polyethylene glycol.

17. The method of claim 16, wherein the second pore-forming agent is a PEG 200.

18. The method of claim 16, wherein the second pore-forming agent is an end-capped PEG.

19. The method of claim 7, wherein the wash solution is an aqueous solution.

20. A dual-porosity membrane fabricated by the method of one of claims 7-19.

21. A method comprising culturing cells on a dual-porosity membrane of one of claim 1-6 or 20.

22. A method comprising administering to a subject cells on or within a dual-porosity membrane of one of claim 1-6 or 20.
